(19) United States
(12) Patent Application Publication
(10) Pub. No.: US 2025/0288196 A1
(43) Pub. Date: Sep. 18, 2025

(54) ENT SPECULUM

(71) Applicant: CLEARWAX LTD, Oadby, Leicester, Leicestershire (GB)

(72) Inventor: Neel RAITHATHA, Oadby, Leicester, Leicestershire (GB)

(73) Assignee: CLEARWAX LTD, Oadby, Leicester, Leicestershire (GB)

(21) Appl. No.: 18/860,106

(22) PCT Filed: Apr. 26, 2023
(86) PCT No.: PCT/GB2023/051112

§ 371 Date: Oct. 25, 2024

(30) Foreign Application Priority Data
Apr. 27, 2022 (GB) 2206142.8

(51) Int. Cl. A61B 1/227; A61B 1/00; A61B 1/32

(52) U.S. Cl. CPC A61B 1/227; A61B 1/00188; A61B 1/32

(57) ABSTRACT

The present invention relates to an ENT speculum comprising a tapered distal portion having a tool access bore therethrough, a proximal portion comprising a connector and having a proximal aperture therethrough, and an intermediate portion intermediate the distal portion and the proximal portion, the distal, intermediate and proximal portions being arranged axially along said longitudinal axis, wherein said proximal aperture and tool access bore are aligned such that they form a first viewing passage, and wherein said intermediate portion comprises a tool entry aperture through which an ENT tool can access the tool access bore without passing through said proximal aperture in the connector.

### TECHNICAL FIELD OF INVENTION

The present invention relates to the field of ENT specula for straightening and dilating orifices during visual inspection and cleaning procedures, for example the removal of earwax.

### BACKGROUND

Earwax is a natural secretion that occurs within the ear. It is estimated that 2.3 million people in the UK alone suffer from earwax impaction each year that requires intervention (NICE, 2018). Symptoms of earwax impaction can range from hearing loss, earache, discomfort, blocked ear, tinnitus, vertigo, occlusion, itchiness and whistling of hearing aids. The removal of earwax is the most common ear, nose, and throat (ENT) procedure performed in primary care in the UK, with approximately 4 million ears treated annually [Guest, 2004]. There are many different treatments of earwax removal available.

A previously common method of removing earwax, called ‘ear syringing’, involved the flushing of water inside the ear using a high-pressured metal ear syringe. This practice is no longer performed in the UK due to the possible risk of perforating the eardrum. Instead, water is now pumped into the ear at controllable rate and pressure using an irrigation machine. This procedure is known as ‘ear irrigation’. Although reducing the risk of perforating the eardrum, the simple action of pumping water into the ear can still nonetheless give rise to this possibility, as well as the increased likelihood of developing an ear infection. Furthermore, both ear syringing and ear irrigation are ‘blind’ techniques, meaning the clinician is unable to visualise the inside of the ear whilst performing the procedure. This is important in order to safely remove earwax and to avoid potentially causing trauma to the ear canal and eardrum itself.

Therefore, the safest and most effective method of treating earwax impaction is to use an array of ‘dry’ ENT micro-instruments (e.g., microsuction, wax hooks, ear curettes, forceps etc.) to manipulate the earwax out of the ear under direct visualisation. There are many different visualisation techniques available, each with their own advantages and disadvantages.

Hand-held operating otoscopes can be used to visualise the inside the ear whilst removing earwax using ENT micro-instruments. A speculum, used to straighten and widen the ear canal, is attached to a mounting system on the otoscope, through which the ENT micro-instrument is also inserted. The clinician can then, with one eye, peer through the magnifying lens of the otoscope. Although low cost and portable, it can be restrictive and limiting to simultaneously look through the magnifying lens with one eye whilst attempting to remove earwax. Moreover, the limited working length available due to the short distance between the magnification lens and speculum mounting system can make it restrictive, and indeed sometimes not possible, to use some ENT micro-instruments. Furthermore, although the speculum can either be disposed of or reused after undergoing the necessary sterilization processes, the mounting system to which the speculum is attached to and through which the ENT micro-instrument is inserted, cannot. As a result, the speculum mounting system can potentially harbour earwax, as well as unwanted and harmful bacteria, viruses and fungi, therefore potentially leading to cross-contamination between individuals. Finally, operating otoscopes only provide low-level magnification.

An operating microscope, on the other hand, can provide very high-level magnification of inside the ear when removing earwax using ENT micro-instruments. A clinician, using their non-dominant hand, uses a speculum to straighten and widen the ear canal, through which they then introduce, with their dominant hand, the ENT micro-instrument inside the ear in order to remove earwax. It also has the added benefit of providing binocular vision to assist in depth perception. However, high-level training is required to be able to use an operating microscope competently. In addition, the cost of an operating microscope can be very expensive. They are also not portable.

Head-worn microscopes are a more cost-effective and portable option than an operating microscope. However, whilst some head-worn microscopes can also provide binocular vision, none offer the same high-level magnification of inside the ear as an operating microscope. Moreover, both operating and head-worn microscopes have a limited optimal focal range and narrow field of view, meaning the clinician will require to constantly reposition themselves and/or the microscope to regain focus if moving on to remove earwax from a different part of the ear and/or if the individual being treated moves.

An endoscope is another method of visualising inside the ear when removing earwax. Not only is it a cost-effective and portable option, it offers good magnification, increased optical focal range and a wide field of view. Since no speculum is being used, the ear canal is instead straightened and widened with the distal end of the endoscope whilst inside the ear.

Unlike an operating otoscope, the eyepiece of an endoscope can also be coupled to a viewing monitor via a camera adapter, allowing for an enhanced and enlarged view of inside the ear. They can also be mounted to portable electronic camera devices, with the image viewable, enlarged and optimised by various apps. An example of this is the Clearwax iCLEARscope®. Such technology also allows for the recording of procedures, useful in potential cases of litigation, training and education purposes and also for advertising and marketing. A limitation, however, of using an endoscope when removing earwax is that some clinicians may struggle with the bilateral hand co-ordination and integration that is necessary when using an endoscope. This is because the non-dominant hand is required at all times to hold the endoscope to view, straighten and widen the inside of the ear canal whilst the dominant hand is simultaneously holding and manipulating the ENT micro-instrument to remove earwax.

The TYMPA™ device developed by TympaHealth utilises the basic principles of an operating otoscope but instead using a portable electronic camera device. This means the clinician can view and optimise an enlarged image of inside the ear on the monitor of the portable electronic device via an app. However, due to the short working length between the back of the portable electronic device and speculum mounting system, no other ENT micro-instruments other than a suction probe can be inserted through the speculum and into the ear, making its application for removing earwax very limited. Additionally, the sub-optimal working length prevents some clinicians from gripping the suction probe by the handle itself. Instead, the connector of the suction probe tends to be used as a makeshift handle. This can of course reduce the clinician's fine motor handling skills with the suction probe inside the ear, which is essential when removing earwax.

Furthermore, the optics and design of the TYMPA™ system can make the suction probe appear distorted and out of focus when in-situ, thus potentially compromising patient care. Moreover, and similarly to an operating otoscope, although the speculum used is disposable, the mounting system to which the speculum is attached and through which the ENT micro-instrument is inserted is not. This can therefore lead to cross-contamination of potential earwax, as well as unwanted and harmful bacteria, viruses and fungi between individuals.

Consequently, it is an object of the present invention to provide a cost-effective and portable device to assist in the removal of earwax, which seeks to address the disadvantages inherent to known devices.

### SUMMARY OF THE INVENTION

An aspect of the invention is set out in the independent claim. Dependent claims describe optional features.

A first aspect of the present invention provides an ENT speculum (100) comprising a longitudinal axis (L) along which is arranged: a tapered distal portion (106) having a tool access bore (106B) therethrough, a proximal portion (102) comprising a connector and having a proximal aperture (102D) therethrough, and an intermediate portion (104) intermediate the distal portion (106) and the proximal portion (102), the distal, intermediate and proximal portions being arranged axially along said longitudinal axis, wherein said proximal aperture and tool access bore are aligned such that they form a first viewing passage, and wherein said intermediate portion (104) comprises a tool entry aperture (104A) through which an ENT tool can access the tool access bore without passing through said proximal aperture in the connector.

Advantageously, said ENT speculum provides a distal portion which can be inserted into the ear such that the ear canal is straightened and widened giving easier access for tools. Once the tip of the distal portion is within the ear, a tool can be inserted into the ear via the tool entry aperture located between the distal portion and the proximal portion and subsequently through the tool access bore. An operative can look through the first viewing passage formed by the proximal aperture and the tool access bore to provide vision whilst they use a tool, such as an ENT tool to remove earwax.

When the proximal portion is held by the operative or attached to a lighting and/or magnification device (such as an otoscope, endoscope or portable electronic camera device), the tool does not need to pass through the aperture in the proximal portion. Thus, when the tool is removed there is no chance of getting earwax or other contaminants from the tool on the proximal portion of the speculum, the hand of the operative or the lighting/magnification device. This is in contrast to prior art specula which require that the tool be inserted through a proximal opening, through a passage, through the distal portion and into the ear, then on retraction of the device the operative needs to take care not to touch the proximal area of the speculum, any specula mounting system, the hand of the operative or the lighting/magnification device with the tool in order to prevent cross contamination.

In an embodiment, said tool entry aperture is configured such that an ENT tool can access the tool access bore at an angle incident to said longitudinal axis.

Providing access at an angle incident to the longitudinal axis increases the viewing area of the inner ear because the handle can be held off to one side. This provides an advantage because the user can see the position of the tool within the ear better. Holding the handle off to one side also provides ergonomic benefits because the handle and the speculum are located spatially away from each other.

ENT tools can be provided with a curved handle, or a straight handle with a curved tool, such that the end of the tool car be held at a point out of the visual field. Therefore, it is a benefit to provide a tool entry aperture which allows the operative to hold the tool at an angle relative to the speculum.

In an embodiment, the intermediate portion is between 20 mm and 50 mm in length. The intermediate portion can be of any nominal length required to perform the said procedure.

In an embodiment, said tool entry aperture is formed as a cut-away portion of the intermediate portion.

In an embodiment, the connector is releasably attachable to an otoscope, endoscope or a portable electronic camera device.

Attaching the speculum to an otoscope, endoscope or a portable electronic camera device increases the visibility in the inner ear by providing lighting and magnification.

In an embodiment, the connector is one of a threaded connector, a push snap fit connector, a clip on friction fit, lock or an adhesive connector.

In an embodiment, the proximal portion comprises radially outwardly protruding protrusions.

The speculum may be releasably attached to other equipment. Providing protrusions on the proximal portion will aid users grip when attaching, rotating and removing the speculum from such equipment.

In an embodiment, the speculum is autoclavable. In another embodiment, the speculum is single-use.

In an embodiment, the distal, intermediate and proximal portions are formed from a single piece of material.

In another embodiment, the distal portion is removable from the intermediate and proximal portions.

It may be desirable to remove the distal portion and replace it with a smaller or larger distal portion or a distal portion which tapers more or less, or simply to replace a distal portion which has become soiled, without the need to replace the entire speculum.

A further aspect of the present invention provides an otoscope comprising: a body comprising a speculum receiving portion for receiving the speculum as described in any of the paragraphs above, an eye piece comprising a viewing aperture which forms a second viewing passage, wherein the proximal portion of the speculum is attachable to the speculum receiving portion such that the first and second viewing passages are aligned.

A further aspect of the present invention provides a portable electronic device comprising a camera, a display and attachment means for attaching the otoscope as described above to the portable electronic device such that the camera is aligned with the second viewing passage.

### BRIEF DESCRIPTION OF THE DRAWINGS

Embodiments of the invention are described below, by way of example only, with reference to the accompanying Figures in which:

FIG. 1A is a perspective view of a speculum according to an aspect of the present invention,

FIG. 1B is a side view of the speculum of FIG. 1A,

FIG. 2 is a proximal end view of the speculum of FIGS. 1A and 1B,

FIGS. 3A-3D are schematic views showing how a speculum according to the present invention is attachable to and operable with an otoscope,

FIG. 4 is a cross sectional side view of a push snap fit type connector between an otoscope and a speculum.

### DETAILED DESCRIPTION

The term “earwax” in the context of the present invention should be interpreted as including secreted earwax as well as foreign objects and other debris which needs to be removed from the ear during a treatment.

The term “otoscope” in the context of the present invention should be interpreted as including all devices which are used to illuminate and/or magnify a patient's ear.

The term “electronic device” and “portable electronic device” refer to any electronic device with a screen and a camera, and include a digital camera with a display, a mobile telephone with a camera, or a tablet device with a screen and a camera.

The term “endoscope” in the context of the present invention should be interpreted as including all devices which are used to provide vision, illuminate and/or magnify the interior of an orifice of a patient.

The terms “tip”, “distal tip and “distal portion” are used interchangeably throughout the specification.

The term “ENT” means ear, nose and throat. “ENT tools” are tools used in examinations or procedures in the ear, nose or throat of a patient.

It is expressly intended that the speculum of the present invention can also be used in veterinary practice, in the ear, nose or throat of animals.

Certain terminology is used in the following description for convenience only and is not limiting. The words ‘right’, ‘left’, ‘lower’, ‘upper’, ‘front’, ‘rear’, ‘upward’, ‘down’, ‘downward’, ‘above’ and ‘below’ designate directions in the drawings to which reference is made and are with respect to the described component when assembled and mounted (e.g. in situ). The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description. The term “distal” refers to those features which are located furthest from the operator i.e. nearest the patient. The term “proximal” refers to those features which are located closest to the operator i.e. furthest from the patient.

Further, as used herein, the terms ‘connected’, ‘attached’, ‘coupled’, ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, ‘first’, ‘second’, ‘third’ etc merely indicate that different instances of like objects are being referred to and are not intended to imply that the objects so described must be in a given sequence, either temporally, spatially, in ranking or in any other manner.

Through the description and claims of this specification, the terms ‘comprise’ and ‘contain’, and variations thereof, are interpreted to mean ‘including but not limited to’, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality, as well as, singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract or drawings), or to any novel one, or any novel combination, of the steps of any method or process so disclosed.

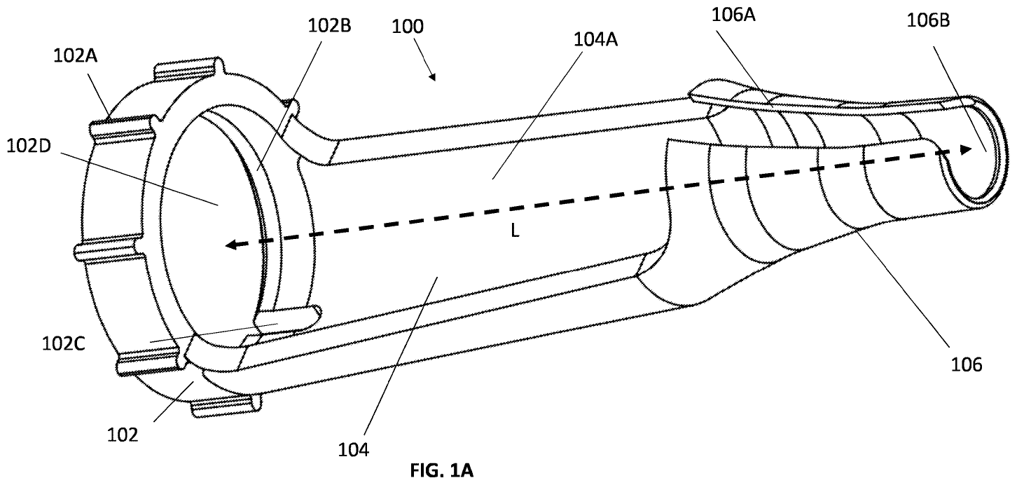

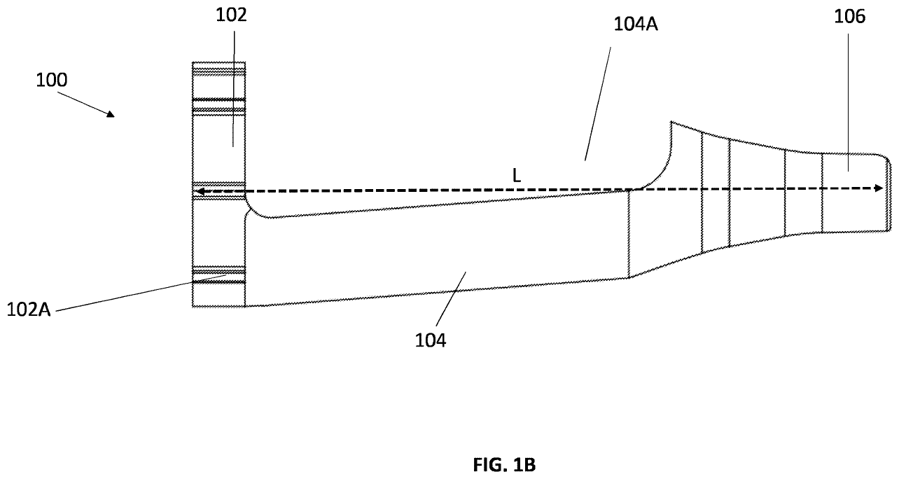

Referring to FIGS. 1A and 1B, the speculum 100 comprises a proximal portion 102, a tapered distal portion 106 and an intermediate portion 104 therebetween, arranged along a longitudinal axis L.

The proximal portion 102 is in the form of an annular connector with a viewing aperture 102D therethrough. The outer surface of the connector is provided with radially outwardly protruding protrusions 102A to aid a user's grip and provides the ability to rotate the speculum when attached to an otoscope or other device to which it is attached to ensure the flexure portion 106A is aligned in the preferred position when inserting the ENT tool. Alternatively, the outer surface may be coated or otherwise textured to improve grip when handling the connector.

The connector is designed to attach to an otoscope or other device via a push snap fit 102B. In other embodiments, the connector may be threaded, clipped on, friction fitted, lock or provided with an adhesive.

The proximal portion 102 includes a locator notch 102C which is used to ensure that the speculum is correctly orientated with respect to the otoscope or other device to which it is attached. The notch 102C may also serve as a rotary endstop if the connector is threaded. The notch further provides flex to the proximal portion 102 such that when being push snap fitted to an otoscope or other device the connector can expand more easily before reverting, thus reducing the force required for attachment. In other embodiments, there may be multiple notches to help provide further flex to the proximal portion 102.

The speculum further comprises a distal portion 106. The distal portion is in the form of a tapered tip with a tool access bore 106B therethrough. The distal portion optionally includes a flexure portion 106A to allow the tip to flex and further dilate the ear canal. In addition, the flexure portion 106A acts as a slot to insert ENT tools into the ear from a higher angle to improve both user visibility and maneuverability.

The speculum 100 further comprises an intermediate portion 104 which is an elongate section intermediate the proximal portion 102 and the distal portion 106. The intermediate portion includes a tool entry aperture 104A which provides access to the tool access bore 106B.

In the embodiment shown the intermediate portion is formed as a C-shaped tube portion or “half pipe” The intermediate portion could be formed as a U-shaped partial pipe or other shape. The intermediate portion could be an almost complete tube with a small section as the tool entry aperture, such that the intermediate portion is a complete tube through more than 350° out of 360° with the tool access aperture comprising less than 10° of the intermediate portion. It is also intended that the opposite could be the case, with the intermediate portion forming less than 10° of the complete tube and the tool access aperture comprising more than 350° of the complete tube. The tool access aperture could comprise anywhere between 1° to 359° including 160°-200° or from 10°-50°, 51°-100°, 101°-150°, 151°-200°, 201°-250°, 251°-300°, 301°-350° of the complete tube with the intermediate portion making up the remainder of the complete tube.

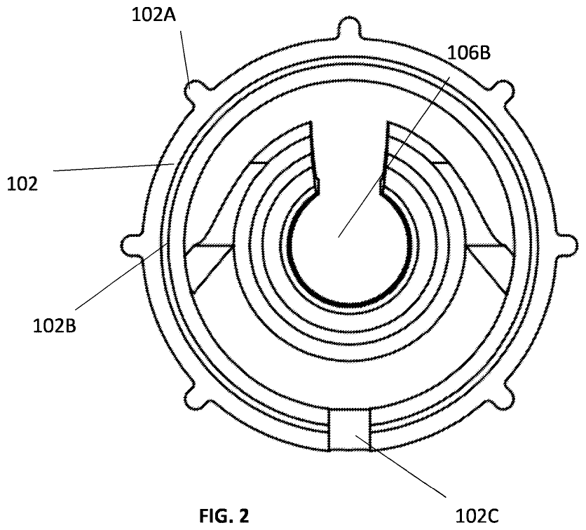

FIG. 2 shows the speculum of FIG. 1A from a different perspective, with the proximal portion nearest to the viewer and the distal portion farthest from the viewer. The radially outwardly protruding protrusions 102A are located equidistantly around the generally circular proximal portion.

The viewing aperture in the proximal portion and the tool access bore 106B through the distal portion are aligned, such that they form a viewing passage. In use the speculum will preferably be used in concert with a magnifying and lighting device such that light is directed down the speculum towards an ear. Alternative uses include providing holding the speculum with a lens attached to the proximal section and providing light via the tool access aperture.

The tool access aperture in the intermediate portion 104 and the tool access bore 106B in the distal portion can be used to provide access to the ear of a patient without requiring the tool to pass through and potentially come into contact with the viewing aperture 102D.

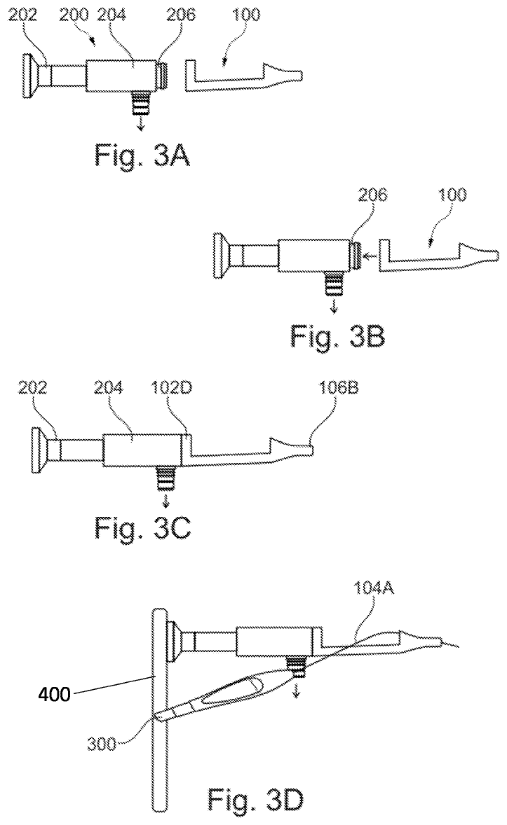

FIG. 3A shows an otoscope 200 and a speculum 100. The otoscope comprises an eyepiece 202, a body 204 and a speculum receiving portion 206. The speculum receiving portion 206 has complementary shape to the connector 102B of the speculum. If the connector 102B is formed as a pushsnap fit, clip on, friction or lock fit connector then the speculum receiving portion 206 will have a complementary shape. If the connector 102B is threaded, the speculum receiving portion 206 will have a complementary external thread.

FIG. 3B shows how a speculum with a push snap fit connector can be secured to the speculum receiving portion 206.

FIG. 3C shows the speculum 100 connected to the otoscope 200. The eyepiece, viewing aperture and tool access bore are all aligned such that they form a viewing passage. Light may be provided to the body 204 via a handle or produced within the body 204 by a light emitter such as an LED bulb. The light is directed down the speculum towards the tool access bore 106B. At least one of the eyepiece, body and speculum may include one or more magnification lenses/optics.

FIG. 3D shows the speculum 100 attached to the otoscope 200 with the otoscope attached to a mobile electronic device 400 with a camera. An ENT tool is also shown. The end of the ENT tool enters the speculum via the tool entry aperture 104A and passes through the tool access bore 106B into a position to work on a patient. The view from the eyepiece 202 is not compromised because the handle of the ENT tool 300 is not obscuring the clinician's view of the ear of the patient. Additionally, as the ENT tool is retracted it will not need to pass close to the otoscope or the proximal portion of the speculum such that the risk of earwax, bacteria, fungi or viruses being transferred to the otoscope is minimised.

The speculum and otoscope shown in FIGS. 3A-3D may also be used with a portable electronic device such as a mobile phone or tablet computer with a camera, as shown in FIG. 3D. The electronic device 400 can be provided with attachment means to connect the speculum, otoscope or endoscope to the electronic device such that the camera is aimed down the eyepiece. The screen of the electronic device can then be used to view the ear of the patient during use of the speculum. The screen of the electronic device can therefore be used to show an enlarged image of the patient's inner ear (or other orifice) and recordings of earwax removal can be taken for training and demonstration purposes.

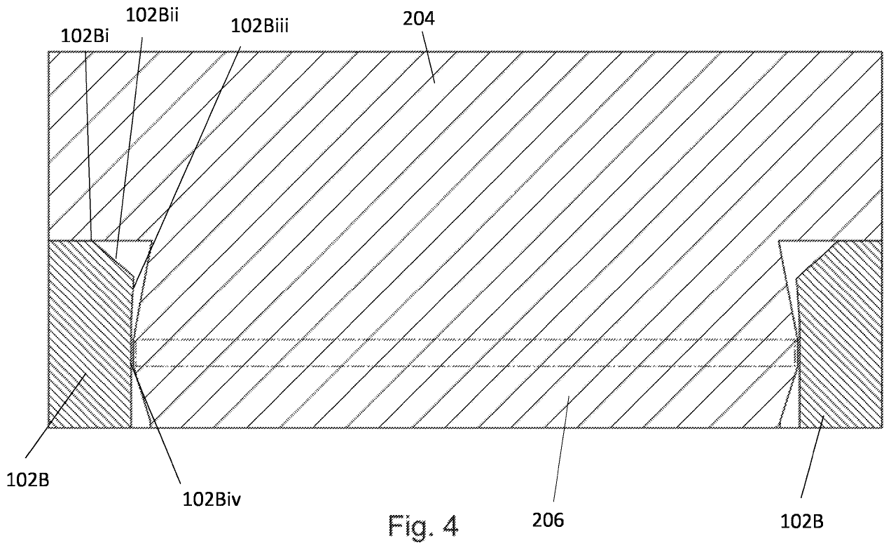

FIG. 4 shows how the proximal portion 102 may connect to a separate device 200. The connector 102B of the proximal portion 102 comprises an annular push snap fit connector comprising a reduced diameter portion 102Bi, a first sloped portion 102Bii with a first gradient, a second sloped portion 102Biii with a second gradient and a parallel portion 102Biv. The separate device has complimentary surfaces such that the speculum can be pushed onto the speculum receiving portion of the separate device. The push snap fit could provide tactile or audible feedback such as a “snap” to confirm that it is correctly located. Preferably some force, achievable by hand, is required to attach and remove the speculum.

It will be appreciated by persons skilled in the art that the above embodiment(s) have been described by way of example only and not in any limitative sense, and that various alterations and modifications are possible without departing from the scope of the invention as defined by the appended claims. Various modifications to the detailed designs as described above are possible, for example, variations may exist in shape, size, arrangement (i.e. a single unitary components or two separate components), assembly or the like.

The following numbered list is a list of clauses which are not to be confused with the claims:

1. An ENT speculum (100) comprising a longitudinal axis (L) along which is arranged:
a tapered distal portion (106) having a tool access bore (106B) therethrough,
a proximal portion (102) comprising a connector and having a proximal aperture (102D) therethrough, and
an intermediate portion (104) intermediate the distal portion (106) and the proximal portion (102),
the distal, intermediate and proximal portions being arranged axially along said longitudinal axis, wherein said proximal aperture and tool access bore are aligned such that they form a first viewing passage, and
   wherein said intermediate portion (104) comprises a tool entry aperture (104A) through which an ENT tool can access the tool access bore without passing through said proximal aperture in the connector.
   2. The speculum of clause 1 wherein said tool entry aperture is configured such that an ENT tool can access the tool access bore at an angle incident to said longitudinal axis.
   3. The speculum of clause 1 or clause 2 wherein said intermediate portion is between 20 mm and 50 mm in length.
   4. The speculum of any of the preceding clauses wherein said tool access aperture is formed as a cut-away portion of the intermediate portion.
   5. The speculum of any of the preceding clauses wherein said connector is releasably attachable to an otoscope, endoscope or a portable electronic camera device.
   6. The speculum of any of the preceding clauses wherein said connector is one of a threaded connector, a push snap fit connector, clip on, friction fit, lock or an adhesive connector.
   7. The speculum of any preceding clause wherein the proximal portion comprises radially outwardly protruding protrusions.
   8. The speculum of any of the preceding clauses wherein the speculum is single-use.
   9. The speculum of any of the preceding clauses wherein the speculum is autoclavable.
   10. The speculum of any of the preceding clauses wherein the distal, intermediate and proximal portions are formed from a single piece of material.
   11. The speculum of any of clauses 1-9 wherein the distal portion is removable from the intermediate and proximal portions.
   12. An otoscope or endoscope comprising:
a body comprising a speculum receiving portion for receiving the speculum of any of clauses 1 to 11;
an eye piece comprising a viewing aperture which forms a second viewing passage;
wherein the proximal portion of the speculum attaches to the speculum receiving portion such that the first and second viewing passages are aligned.
   13. A portable electronic device comprising a camera, a display and attachment means for attaching the speculum of any of clauses 1-11 or the otoscope or endoscope of clause 12 to the portable electronic device such that the camera is aligned with the second viewing passage.

1. An otoscope comprising:
a body comprising one or more optics and a speculum receiving portion at a first end of said body;
an ENT speculum connectable to the speculum receiving portion, the speculum comprising a longitudinal axis along which is arranged:
a tapered distal portion having a tool access bore therethrough,
a proximal portion comprising a connector and having a proximal aperture therethrough, and
an intermediate portion intermediate the distal portion and the proximal portion,
the distal, intermediate and proximal portions being arranged axially along said longitudinal axis, wherein said proximal aperture and tool access bore are aligned such that they form a first viewing passage, and
said otoscope further comprising:
an optical output connected to a second end of the body, comprising a viewing aperture which forms a second viewing passage;
a handle attachable to the body and comprising a light emitter;
wherein said intermediate portion comprises a tool entry aperture through which an ENT tool can access the tool access bore without passing through said proximal aperture in the connector;
wherein the proximal portion of the speculum attaches to the speculum receiving portion such that the first and second viewing passages are aligned.

2. The otoscope of claim 1 wherein said tool entry aperture is configured such that an ENT tool can access the tool access bore at an angle incident to said longitudinal axis.

3. The otoscope of claim 1 wherein said intermediate portion is between 20 mm and 50 mm in length.

4. The otoscope of claim 1, wherein said tool access aperture is formed as a cut-away portion of the intermediate portion.

5. The otoscope of claim 1, wherein said connector is one of a threaded connector, a push snap fit connector, clip on, friction fit, lock or an adhesive connector.

6. The otoscope of claim 1, wherein the proximal portion of the speculum comprises radially outwardly protruding protrusions.

7. The otoscope of claim 1, wherein the speculum is single-use.

8. The otoscope of claim 1, wherein the speculum is autoclavable.

9. The otoscope of claim 1, wherein the distal, intermediate and proximal portions are formed from a single piece of material.

10. The otoscope of claim 1, wherein the distal portion is removable from the intermediate and proximal portions.

11. The otoscope of claim 1 wherein the optical output comprises an eyepiece and/or an electronic device comprising a camera and a display.

12. The otoscope of claim 1, wherein at least one of the eyepiece, body and speculum further comprise one or more magnification lenses or optics.

13. A portable electronic device comprising a camera, a display and attachment means for attaching the otoscope of claim 1, to the portable electronic device such that the camera is aligned with the second viewing passage.

14. A kit of parts comprising:
a body comprising one or more optics and a speculum receiving portion at a first end of said body,
an ENT speculum connectable to the speculum receiving portion, the speculum comprising a longitudinal axis along which is arranged:
a tapered distal portion having a tool access bore therethrough,
a proximal portion comprising a connector and having a proximal aperture therethrough, and
an intermediate portion intermediate the distal portion and the proximal portion,
the distal, intermediate and proximal portions being arranged axially along said longitudinal axis, wherein said proximal aperture and tool access bore are aligned such that they form a first viewing passage, and
an optical output connectable to a second end of the body, comprising a viewing aperture which forms a second viewing passage;
a handle attachable to the body and comprising a light emitter;
wherein said intermediate portion comprises a tool entry aperture through which an ENT tool can access the tool access bore without passing through said proximal aperture in the connector;
wherein when the handle is connected to the body, light produced by the light emitter is directed down the speculum towards the tool access bore by said optics;
wherein the proximal portion of the speculum attaches to the speculum receiving portion such that the first and second viewing passages are aligned.
